SEQUENCE LISTING (49 sequences)

SEQ ID NO: 1 - DNA, 1419 bp, Artificial Sequence
  Heavy chain counter selection marker
tggagcccttccttgttacttcataccatcctctgtgcttccttcctcagggcctggtgatgatggcgggatcgttgtatatttcttgacaccttttcggcatcgccctaaaattcggcgtcctcatattgtgtgaggacgttttattacgtgtttacgaagcaaaagctaaaaccaggagctatttaatggcaacagttaaccagctggtacgcaaaccacgtgctcgcaaagttgcgaaaagcaacgtgcctgcgctggaagcatgcccgcaaaaacgtggcgtatgtactcgtgtatatactaccactcctaaaaaaccgaactccgcgctgcgtaaagtatgccgtgttcgtctgactaacggtttcgaagtgacttcctacatcggtggtgaaggtcacaacctgcaggagcactccgtgatcctgatccgtggcggtcgtgttaaagacctcccgggtgttcgttaccacaccgtacgtggtgcgcttgactgctccggcgttaaagaccgtaagcaggctcgttccaagtatggcgtgaagcgtcctaaggcttaaggaggacaatcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaagacaaaggtcctgagacgccaccaccagctccccagctccatcctatct

SEQ ID NO: 2 - DNA, 8293 bp, Artificial Sequence
  Heavy chain knock-in sequence
aattcagacatcatgcagggccagttccctgccctgagctacattagctgggaccagggccagcggttgaggaaccaggcagaggtaaaatggtggtgtgataggaagccaatggcagaggggagggagaagttatgcttatgtcatgctggaatgtaggaaggggaaagagccaggatgtctaggctggagctgatccggctgtctgctctgatggcagcaacaggcctgagcttctctggactcaagaagccagggcaacaaaataaagggggcctagcagagcaaagacactgctagcactgggatcaggaaaacaggacaagactcccgatccaggaggtcatgggagggaaggagaagactacaggggactgtccttgggaaagagtaagggcccactggagggagtgctcaggaagcaagcccattgacaggggagaacaaggctggtggacgtctggatgggcagtaggcagccccaagtcccaggagggagagaagaggcagataggaaaacaggtcaggtctagcagaggcctactgaagtactctcctcaggacagaaccctgaatactggaaaatgcggaactgctgcaggcacaaagaatagctgaggtctaagagtaaaacagactaggggatgagaggaccttaggaagagcctttggctgagcaggaacaagaacaggggaaatcctagggctgacattgccagtggaaacatacaggctggagctctttagtcaggagctccagctgtgatctagacatcaggcaggaagatcaaatctgtcccaacaatacaggggacagaggctcaacctagagtgtgagcatcaggggctgtgcaggagatttcagagctcaggtgcagcagagactagcatggccctggggataaagggaaggatccaagggacaaggggataatcctggggaggtaagggccagcttcgtgacagaaggtggtggtgtccaacttcaagagccctgtgctacaatttaaaaaaaaaaaaaaaggaaagggacttctctgtgtttggcaacacaagtgcgatgcacaggcaggaagatcaaatctgtcccaacaatacaggggacagagggtcaacctacaaaaggaaagaacctggggcagtgtgaagacaacactgtagaagccaaggctgagttcactgagctctcgttagtgagactacacagcaaggaggtggcgggcactgagcagtgaggccccgggaagtgggggtgatggtggtgacggtggtaactgttaagaactgggggaaagaattgtggagaaccaagctaaatagttatgtcaaaccacatgtttaggagcctgggttgacttcatagggagtaggcatggaggctaatctagaggtttgtgtataggcaagaagtgaatcctgacccaagaatagagagtgctaaacggacttagttcaaagacaactgaaaaagacaatgcctgcaaaacaaagctaaggccagagctcttggactatgaagagttcagggaacctaagaacagggaccatctgtgtacaggccaaggccggtagaagcagcctaggaagtgtcaagagccaacgtggctgggtgggcaaagacaggaagggactgttaggctgcagggatgtgccgacttcaatgtgcttcagtattgtccagattgtgtgcagccatatggcccaggtataagaggtttaacagtggaacacagatgcccacatcagacagctggggggcgggggtgaacacagatacccatactggaaagcaggtggggcattttcctaggaacgggactgggctcaatggcctcaggtctcatctggtctggtgatcctgacattgataggcccaaatgttggatatcacctactccatgtagagagtcggggacatgggaagggtgcaaaagagcggccttctagaaggtttggtcctgtcctgtcctgtctgacagtgtaatcacatatactttttcttgtagcctccacacagagcccatccgtcttccccttgacccgctgctgcaaaaacattccctccaatgccacctccgtgactctgggctgcctggccacgggctacttcccggagccggtgatggtgacctgggacacaggctccctcaacgggacaactatgaccttaccagccaccaccctcacgctctctggtcactatgccaccatcagcttgctgaccgtctcgggtgcgtgggccaagcagatgttcacctgccgtgtggcacacactccatcgtccacagactgggtcgacaacaaaaccttcagcggtaagagagggccaagctcagagaccacagttcccaggagtgccaggctgagggctggcagagtgggcaggggttgagggggtgggtgggctcaaacgtgggaacacccagcatgcctggggacccgggccaggacgcgggggcaagaggagggcacacagagctcagagaggccaacaaccctcatgaccaccagctctcccccagtctgctccagggacttcaccccgcccaccgtgaagatcttacagtcgtcctgcgacggcggcgggcacttccccccgaccatccagctcctgtgcctcgtctctgggtacaccccagggactatcaacatcacctggctggaggacgggcaggtcatggacgtggacttgtccaccgcctctaccacgcaggagggtgagctggcctccacacaaagcgagctcaccctcagccagaagcactggctgtcagaccgcacctacacctgccaggtcacctatcaaggtcacacctttgaggacagcaccaagaagtgtgcaggtacgttcccacctgccctggtggccgccacggaggccagagaagaggggcgggtgggcctcacacagccctccggtgtaccacagattccaacccgagaggggtgagcgcctacctaagccggcccagcccgttcgacctgttcatccgcaagtcgcccacgatcacctgtctggtggtggacctggcacccagcaaggggaccgtgaacctgacctggtcccgggccagtgggaagcctgtgaaccactccaccagaaaggaggagaagcagcgcaatggcacgttaaccgtcacgtccaccctgccggtgggcacccgagactggatcgagggggagacctaccagtgcagggtgacccacccccacctgcccagggccctcatgcggtccacgaccaagaccagcggtgagccatgggcaggccggggtcgtgggggaagggagggagcgagtgagcggggcccgggctgaccccacgtctggccacaggcccgcgtgctgccccggaagtctatgcgtttgcgacgccggagtggccggggagccgggacaagcgcaccctcgcctgcctgatccagaacttcatgcctgaggacatctcggtgcagtggctgcacaacgaggtgcagctcccggacgcccggcacagcacgacgcagccccgcaagaccaagggctccggcttcttcgtcttcagccgcctggaggtgaccagggccgaatgggagcagaaagatgagttcatctgccgtgcagtccatgaggcagcaagcccctcacagaccgtccagcgagcggtgtctgtaaatcccggtaaatgacgtactcctgcctccctccctcccagggctccatccagctgtgcagtggggaggactggccagaccttctgtccactgttgcaatgaccccaggaagctacccccaataaactgtgcctgctcagagccccaggtacacccattcttgggagcgggcagggctgtgggcaggtgcatcttggcacagaggaatgggccccccaggaggggcagtgggaggaggtgggcagggctgagtccccccaggagaggcggtgggaggaggtgggcagggctgaggtgccactcatccatctgccttcgtgtcagggttatttgtcaaacagcatatctgcagggactcatcacagctaccccgggccctctctgcccccactctgggtctaccccctccaaggagtccaaagacccaggggaggtcctcagggaaggggcaagggagcccccacagccctctctcttgggggcttggcttctacccccctggacaggagcccctgcacccccaggtatagatgggcacacaggcccctccaggtggaaaaacagccctaagtgaaacccccacacagacacacacgacccgacagccctcgcccaagtctgtgccactggcgttcgcctctctgccctgtcccgccttgccgagtcctggccccagcaccggggccggtggagccgagcccactcacaccccgcagcctccgccaccctgccctgtgggcacaccaggcccaggtcagcagccaggccccctctcctactgccccccaccgccccttggtccatcctgaatcggcccccaggggatcgccagcctcacacacccagtctcgcccactcacgcctcactcaaggcacagctgtgcacacactaggccccatagcaactccacagcaccctgtaccaccaccagggcgccatagacaccccacacgtggtcacacgtggcccacactccgcctctcacgctgcctccagcgaggctactgccaagcccttcctctgagccatacctgggccgctggatcccagagagaaatggagaggccctcacgtggtgtcctccagtccaaccctccctgtcaccctgtcagcagcagcaccccacagccaaacacaggatggatgcgtgggctccatcccccactcacccacaccggaaccccagagcaggctacgtgcccctcacagacctcaaacccacatgtgcatctgacaccccagatccaaacgctccccccggtcatgcacaccaagggcacagcacccaccaaatccacacggaaacacgggcaccgggcaccccatgagcacaaagcccctccatgtctgaagacagtccctgcacaccgtcacagccatacattcagcttcactctcacgtcccagcccacctgcacccagctctgggcctggagcagcagaaagaggtgtgagggcccgaggcgggacctgcacctgctgatgacccgggaccagcaggcagctcacggtgttggggaagggagtggagggcacccagggcaggagccagagggaccaggctggtgggcggggccgggccggggtagggccaggaggcagctctggacacccacaggcctgggctcatagtccacaccaggacagcccctcagagcacccatgcagtgagtcccaggtcttgggagccaggccgcagagctcacgcatccttccgagggccctgagtgaggcggccactgctgtgccgaggggttgggtccttctctggggagggcgtggggtctagagaggcggagtggaggtaaccagaggtcaggagagaagccgtaaggaacagagggaaaatggggccagagtcggggcgcagggacgagaggtcaggagtggtcggcctggctctgggccgttgactgactcgggacctgggtgcccaccctcagggctggctggcggctccgcgcagtcccagagggccccggatagggtgctctgccactccggacagcagcagggactgccgagagcagcaggaggctctgtcccccacccccgctgccactgtggagccgggagggctgactggccaggtcccccagagctggacgtgtgcgtggaggaggccgagggcgaggcgccgtggacgtggaccggcctctgcatcttcgccgcactcttcctgctcagcgtgagctacagcgccgccatcacgctcctcatggtgggcacccacctccaggggcccggccagggcagggggttgggcagagccagcagagcgccctgacccacgccctcccctcaggtgcagcggttcctctcagccacgcggcaggggaggccccagacctccctcgactacaccaacgtcctccagccccacgcctaggccgcgggccacctcctgtaatggcatttcccaggccccgaaggaccctgtccaatatgccaagcagcacaactgagatcacactgtctgctcatctcgctttcctccgaccccgagactcagctactctcaaattttccctctctgaaggaccatgtggacattacattgctccaggccacagccaccaggacctaaaacaccatcacagcagcaccaaagacactggatagacccacaagggcaatagtttcctcaacagtatatccaaactgttgggacaaacgagcaatcactgaagaagtgacaagttcccacaatgtcagtgtccagctgagaagggacaaaaagtggtaccagccctgtccacaccaccttctaattcacaggaatacgtgatagaagaggcaggttgtagatccgaaagatgagacagattttatcaactccagaaagagctgggtccaactgaattattctagcgaccttggcattgtcatgacctgccatgaccttcctccttaacacttcgataaaccctgggatatggaaaatgcctgtgtttctcagggtttgggaaagaaccatccatgttgggattcttgtgtagatcctccttctggtcacagatgcaatacactggattttcaggcaaaggagcaaattcacagacaactctggccctacagccctaagacctagacaccaccatctccttggaattatcaaatttaacacccggcacacaacaaagaaggactgggactttgaggcctttgtgtagccctagagggggcagaggccactgagcagggattgggtgatcacaaggacctcctggagagggacctgaggagcaggttccaattgggccaaagaaagaagaacaacaatagagatgaaggatgctggaaagagccatggtacagcagtcttgtccttcagacatgactcttacagcccaggactcttacagtagctagctggagcagaagtccaagggattaccatgccctagggccacaggctactggagggtggagtgagtctactacacaggtccaatgcctgtttctccattgcttctcagccaatgagaaatcagagtctccacctccaagaaaaaggaaggtggaaatgaaaggtgagcacctgccttcccgtgactggcagaaagatctccacggactcaaggctttgacttcaaacttcccgtagatccctgatgtctttaaggactctgtctgctttgttggttttgtttgtttgttttgtactttgtcaataaaacatttttcaaatattctacaattgctgtgctcttcctatgcaaactggccctggcacttcaaaacatggcacagttaagttgaccagtgggccatgcagagcatactacccctcctggtcctgtgtcctaccagatatcctctaagtgtcccttcactgctgggctccagttctgctgccctgaaccacacaggtcctcagtctgtcctccctatgggcagtttcatcccagccagaagctgccctgtggcccctaggctgcccaggcatggtctcccacaccaaccacacaaactaagaagcctgtcccatacattgacctcaggctcagtgataacatttctatagaaaaaggaaaggaataagaaaaaaaaaactccatattacatgggggctgcctaccttgctgatgcttattgtctcaagcaagtttggagaagttccaaatcaggcactgacagggtgggttctcagccatgctcttcttacctggtaggtgccttctcccccatggcacctcacaggctctccatctgtgtgtgtctgggtcctgatctcttctcataagtacaaagtcaggctggaagaggtacaccctagccctcattataacttaccagtttatgatcctgtctgcaaacatctgaggtccctgtggctcaaatgtcaattagtgagtcttctggggacagaatttagtccacattagctctccctgtggaaagtactgcctacatcctatgcccctccccccatggccacttcccaaggtctcatccatccagcatcagctgtctcaagtctcctccaaagctctgctagttaggtaagggtgagatgtgggtgtaactcattcaggggcaagctcacattgtataggccatggctgtcaggaatgtcattaaggctgtcagcacagagaagacacttattccaggttgatctgagcaggtgctgagc

SEQ ID NO: 3 - DNA, 1613 bp, Artificial Sequence
  Heavy chain selection marker
tatctcagagttcaagtgtacagtccccgaagcaagagtcttgccttcttctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttccactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcagcacgtgttgacaattaatcatcggcatagtatatcggcatagtataatacgacaaggtgaggaactaaaccatgggatcggccattgaacaagatggattgcacgcaggttctccggccgcttgggtggagaggctattcggctatgactgggcacaacagacgatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgacgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgcccccgattcgcagcgcatcgccttctatcgccttcttgacgagttcttctgagcgggactctggggttcgaataaagaccgaccaagcgacgtctgagagctccctggcgaattcggtaccaataaaagagctttattttcatgatctgtgtgttggtttttgtgtgcggcgcgttctgttgcatcctgacaatagcagttttcctcaatggcagtttaagcct

SEQ ID NO: 4 - DNA, 1419 bp, Artificial Sequence; identical to SEQ ID NO: 1
  Light chain counter selection marker

SEQ ID NO: 5 - DNA, 423 bp, Artificial Sequence
  Light chain knock-in sequence
tggagcccttccttgttacttcataccatcctctgtgcttccttcctcaggaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagagacaaaggtcctgagacgccaccaccagctccccagctccatcctatct

SEQ ID NO: 6 - DNA, 1716 bp, Artificial Sequence
  Light chain selection marker
ttcttcccctaagtcgtcaaagtcctgaagggggacagtctttacaagcagggcgaattgggccgcataacttcgtatagcatacattatacgaagttatattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttccactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcagcacgtgttgacaattaatcatcggcatagtatatcggcatagtataatacgacaaggtgaggaactaaaccatgggatcggccattgaacaagatggattgcacgcaggttctccggccgcttgggtggagaggctattcggctatgactgggcacaacagacgatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgacgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgcccccgattcgcagcgcatcgccttctatcgccttcttgacgagttcttctgagcgggactctggggttcgaataaagaccgaccaagcgacgtctgagagctccctggcgaattcggtaccaataaaagagctttattttcatgatctgtgtgttggtttttgtgtgcggcgcgataacttcgtatagcatacattatacgaagttatctcgatagcttgagtacatgttctgtaatctgattcaacctacccagtaaacttggcgaagcaaag

SEQ ID NO: 7 - DNA, 937 bp, Artificial Sequence
  Dig-labelled probe
gggataatcctggggaggtaagggccagcttcgtgacagaaggtggtggtgtccaacttcaagagccctgtgctacaatttaaaaaaaaaaaaaaaggaaagggacttctctgtgtttggcaacacaagtgcgatgcacaggcaggaagatcaaatctgtcccaacaatacaggggacagagggtcaacctacaaaaggaaagaacctggggcagtgtgaagacaacactgtagaagccaaggctgagttcactgagctctcgttagtgagactacacagcaaggaggtggcgggcactgagcagtgaggccccgggaagtgggggtgatggtggtgacggtggtaactgttaagaactgggggaaagaattgtggagaaccaagctaaatagttatgtcaaaccacatgtttaggagcctgggttgacttcatagggagtaggcatggaggctaatctagaggtttgtgtataggcaagaagtgaatcctgacccaagaatagagagtgctaaacggacttagttcaaagacaactgaaaaagacaatgcctgcaaaacaaagctaaggccagagctcttggactatgaagagttcagggaacctaagaacagggaccatctgtgtacaggccaaggccggtagaagcagcctaggaagtgtcaagagccaacgtggctgggtgggcaaagacaggaagggactgttaggctgcagggatgtgccgacttcaatgtgcttcagtattgtccagattgtgtgcagccatatggcccaggtataagaggtttaacagtggaacacagatgcccacatcagacagctggggggcgggggtgaacacagatacccatactggaaagcaggtggggcattttcctaggaacgggactgggctcaatggcctcaggtctcatctggtctggtgatcctgaca

SEQ ID NO: 8 - DNA, 74 bp, Artificial Sequence
  Heavy chain rpsl-neo cloning forward primer
gtcctgtcctgtcctgtctgacagtgtaatcacatatactttttcttgtaggcctggtgatgatggcgggatcg

SEQ ID NO: 9 - DNA, 74 bp, Artificial Sequence
  Heavy chain rpsl-neo cloning reverse primer
tattggacagggtccttcggggcctgggaaatgccattacaggaggtggctcagaagaactcgtcaagaaggcg

SEQ ID NO: 10 - DNA, 26 bp, Artificial Sequence
  Mouse Cgamma1 cloning forward primer
gaattcagacatcatgcagggccagt

SEQ ID NO: 11 - DNA, 26 bp, Artificial Sequence
  Mouse Cgamma1 cloning reverse primer
atcgattacaagaaaaagtatatgtg

SEQ ID NO: 12 - DNA, 26 bp, Artificial Sequence
  Human Cepsilon cloning forward primer
atcgatgcctccacacagagcccatc

SEQ ID NO: 13 - DNA, 18 bp, Artificial Sequence
  Human Cepsilon cloning reverse primer
aaagctgggcctggtgga

SEQ ID NO: 14 - DNA, 20 bp, Artificial Sequence
  Mutagenesis forward primer
gcctccacacagagcccatc

SEQ ID NO: 15 - DNA, 20 bp, Artificial Sequence
  Mutagenesis reverse primer
tacaagaaaaagtatatgtg

SEQ ID NO: 16 - DNA, 24 bp, Artificial Sequence
  Mouse Cgamma1 cloning forward primer
ccgcgggccacctcctgtaatggc

SEQ ID NO: 17 - DNA, 24 bp, Artificial Sequence
  Mouse Cgamma1 cloning reverse primer
ctcgagctcagcacctgctcagat

SEQ ID NO: 18 - DNA, 70 bp, Artificial Sequence
  Heavy chain neo cloning forward primer
tgggtccaactgaattattctagcgaccttggcattgtcatgacctgccagggcgaattgggccgcataa

SEQ ID NO: 19 - DNA, 70 bp, Artificial Sequence
  Heavy chain neo cloning reverse primer
aggcattttccatatcccagggtttatcgaagtgttaaggaggaaggtcatactcaagctatcgagataa

SEQ ID NO: 20 - DNA, 74 bp, Artificial Sequence
  Light chain rpsl-neo cloning forward primer
atggagcccttccttgttacttcataccatcctctgtgcttccttcctcaggcctggtgatgatggcgggatcg

SEQ ID NO: 21 - DNA, 74 bp, Artificial Sequence
  Light chain rpsl-neo cloning reverse primer
agataggatggagctggggagctggtggtggcgtctcaggacctttgtcttcagaagaactcgtcaagaaggcg

SEQ ID NO: 22 - DNA, 69 bp, Artificial Sequence
  Mouse Ckappa cloning forward primer
atggagcccttccttgttacttcataccatcctctgtgcttccttcctcaggaactgtggctgcaccat

SEQ ID NO: 23 - DNA, 69 bp, Artificial Sequence
  Mouse Ckappa cloning reverse primer
agataggatggagctggggagctggtggtggcgtctcaggacctttgtctctaacactctcccctgttg

SEQ ID NO: 24 - DNA, 70 bp, Artificial Sequence
  Mouse neo cloning forward primer
ttcttcccctaagtcgtcaaagtcctgaagggggacagtctttacaagcagggcgaattgggccgcataa

SEQ ID NO: 25 - DNA, 70 bp, Artificial Sequence
  Mouse neo cloning reverse primer
ctttgcttcgccaagtttactgggtaggttgaatcagattacagaacatgtactcaagctatcgagataa

SEQ ID NO: 26 - DNA, 20 bp, Artificial Sequence
  Heavy chain rpsl-neo screening forward primer
gcctcaggtctcatctggtc

SEQ ID NO: 27 - DNA, 20 bp, Artificial Sequence
  Heavy chain rpsl-neo screening reverse primer
cttggaacgagcctgcttac

SEQ ID NO: 28 - DNA, 20 bp, Artificial Sequence
  Heavy chain rpsl-neo screening forward primer
gcctcaggtctcatctggtc

SEQ ID NO: 29 - DNA, 20 bp, Artificial Sequence
  Heavy chain rpsl-neo screening reverse primer
ggttttgttgtcgacccagt

SEQ ID NO: 30 - DNA, 20 bp, Artificial Sequence
  Human Cepsilon screening forward primer
cactgtgcaaccaaacatcc

SEQ ID NO: 31 - DNA, 20 bp, Artificial Sequence
  Heavy chain neo screening reverse primer
tgtccatctgcacgagacta

SEQ ID NO: 32 - DNA, 18 bp, Artificial Sequence
  Light chain neo screening forward primer
ccctccaatcgggtaact

SEQ ID NO: 33 - DNA, 18 bp, Artificial Sequence
  Light chain neo screening reverse primer
gtttggagcaccgcaaca

SEQ ID NO: 34 - DNA, 20 bp, Artificial Sequence
  Mouse Cgamma1 screening forward primer
cctggccccattgttcctta

SEQ ID NO: 35 - DNA, 20 bp, Artificial Sequence
  Light chain neo screening forward primer
tccactacatggcagtcctt

SEQ ID NO: 36 - DNA, 20 bp, Artificial Sequence
  Heavy chain genotyping forward primer
gaaggtttggtcctgtcctg

SEQ ID NO: 37 - DNA, 22 bp, Artificial Sequence
  Heavy chain genotyping reverse primer
gctgctcagagtgtagaggtca

SEQ ID NO: 38 - DNA, 20 bp, Artificial Sequence
  Heavy chain genotyping reverse primer
ggttttgttgtcgacccagt

SEQ ID NO: 39 - DNA, 20 bp, Artificial Sequence
  Light chain genotyping forward primer
ggcagtgaacgacaaaatgg

SEQ ID NO: 40 - DNA, 20 bp, Artificial Sequence
  Light chain genotyping reverse primer
cctggccccattgttcctta

SEQ ID NO: 41 - DNA, 18 bp, Artificial Sequence
  Light chain genotyping reverse primer
gtttggagcaccgcaaca

SEQ ID NO: 42 - DNA, 20 bp, Artificial Sequence
  Dig-labelled probe forward primer
gggataatcctggggaggta

SEQ ID NO: 43 - DNA, 20 bp, Artificial Sequence
  Dig-labelled probe reverse primer
tgtcaggatcaccagaccag

SEQ ID NO: 44 - DNA, 20 bp, Artificial Sequence
  Mouse Cgamma1 forward primer
atggcaaggagttcaaatgc

SEQ ID NO: 45 - DNA, 20 bp, Artificial Sequence
  Mouse Cgamma1 reverse primer
tttatccttggccatctgct

SEQ ID NO: 46 - DNA, 18 bp, Artificial Sequence
  Human Cepsilon forward primer
tctaccacgcaggagggt

SEQ ID NO: 47 - DNA, 18 bp, Artificial Sequence
  Human Cepsilon reverse primer
ctgtcctcaaaggtgtga

SEQ ID NO: 48 - DNA, 19 bp, Artificial Sequence
  Mouse beta-actin forward primer
gactacctatgaagatcct

SEQ ID NO: 49 - DNA, 20 bp, Artificial Sequence
  Mouse beta-actin reverse primer
ccacatctgctggaaggtgg

(19) United States
(12) Patent Application Publication
(10) Pub. No.: US 2017/0049084 A1
(43) Pub. Date: Feb. 23, 2017

(54) TRANSGENIC ANIMALS CAPABLE OF PRODUCING HUMANIZED IGE AT MUCH HIGHER LEVELS THAN MOUSE IGE

(71) Applicant: ALLERMABS CO. LTD., Taipei City (TW)

(72) Inventors: Alfur Fu-Hsin HUNG, Taichung City (TW); Donic Chien-Sheng LU, Kaohsiung City (TW); Tse-Wen CHANG, Taipei (TW)

(73) Assignee: ALLERMABS CO. LTD., Taipei City (TW)

(21) Appl. No.: 15/110,607

(22) PCT Filed: Jan. 21, 2015
(86) PCT No.: PCT/CN2015/071264

§ 371 Date: Jul. 8, 2016

Related U.S. Application Data
(60) Provisional application No. 61/925,836, filed on Jan. 10, 2014.

(51) Int. Cl. A01K 67/027; C07K 16/00

(52) U.S. Cl. CPC A01K 67/0278; C07K 16/00; A01K 2267/01; A01K 2227/105; A01K 2217/072; C07K 2317/24; C07K 2317/52

(57) ABSTRACT

Provided are transgenic non-human animals, in whose genome the coding sequence of one of the endogenous immunoglobulin Cγ constant regions is replaced by a human immunoglobulin Cε constant regions coding sequence. One preferred transgenic animal is a mouse, in whose genome the Cγ1 constant regions are replaced by human immunoglobulin Cε constant regions and the Cκ constant region is replaced by human immunoglobulin Cκ constant region. The transgenic mouse yields humanized IgE-secreting B cells and antigen-specific humanized IgE after immunization. Also provided are the applications of the transgenic animals for preparing serum which containing humanized IgE, antiserum which containing humanized IgE, and monoclonal antigen-specific humanized IgE antibodies by hybridoma or other technologies.

### BACKGROUND AND RATIONALE

IgE plays a central role in mediating type I hypersensitivity reactions that are responsible for causing allergic diseases, including allergic asthma, allergic rhinitis, atopic dermatitis, and others. Allergic reactions result from the immune response to harmless environmental substances, such as dust mites, tree and grass pollens, certain foods, insect stings, and others. In sensitized individuals, the immune system produces IgE specific to the antigens the persons are sensitized to. In an allergic reaction, the antigen inhaled, ingested, or taken in through the skin by a sensitized person binds to IgE on the surface of basophils and mast cells, thus causing the cross-linking of the IgE and the aggregation of the underlying receptor of IgE.Fc (the type I IgE.Fc receptor, or FcεRI), leading to the release of pharmacologic mediators, such as histamine, leukotrienes, tryptase, cytokines and chemokines from those inflammatory cells. The release of those mediators from mast cells and basophils causes the various pathological manifestations of allergy.

The genes encoding the classes and subclasses of immunoglobulins, including the constant regions of μ, δ, γ, α, and ε chains, are clustered in a stretch of coding regions and introns in one chromosome in the respective genome of human, mouse, or other mammals. In both humans and mice, there are several γ subclasses and one functional ε subclass. The expression and stability of Ig classes and subclasses are regulated by a host of regulatory factors and receptors expressed by B and T lymphocytes and other cell types and by a large array of segments/elements of DNA in the genes of the immunoglobulins.

Among the five Ig classes, IgE is generally present in minute concentrations in serum in non-atopic persons, generally ranging from 10 to 400 ng/ml (Hellman 2007). The concentrations of IgE in mice, rats, rabbits, and other mammals are also very low compared to IgG, IgM, and IgA. In the preparation of mouse or rat hybridomas, which secrete monoclonal antibodies specific for the antigens used in immunizing the animal hosts, hybridomas secreting IgE are extremely rare and very difficult to obtain. In contrast, IgG is the dominant plasma Ig class with serum concentrations normally in the range of 8˜16 mg/ml (Hellman 2007). In preparing mouse or rat hybridomas, IgG is the dominant class of antibodies the hybridomas secrete.

Hybridomas secreting hapten-, ovalbumin-, or allergen component-specific mouse IgE can be prepared by fusing splenocytes from antigen-immunized mice or rats with a mouse myeloma cell line by a conventional cell fusion technique (Bottcher 1980, Bohn 1982, Akihiro 1996, Hanashiro 1996, Susanne 2003). Typically not a single antigen-specific IgE hybridoma can be identified even from several hundreds of hybridoma clones, most of which secret IgG isotypes. The Yu's group constructed an IgE knock-in mouse line in which the DNA sequence encoding mouse Ig γ1 constant region was replaced by the sequence encoding mouse Ig ε constant region (Yu 2013). Total serum IgE levels in those mice increased about ten folds as compared to those in the wild type mice. The number of IgE-expressing lymphocytes isolated from the spleen of a knock-in mouse also significantly increased under the stimulation with lipopolysaccharide (LPS) and Interleukin-4 (IL-4) in vitro. The Zarrin's group constructed an SμKI mouse line in which the switch region of Ig ε heavy chain gene was substituted by the switch region of mouse Ig μ heavy chain gene (Zarrin, 2013). A switch region is a conserved DNA sequence upstream of Ig heavy chain gene and plays a role in Ig isotype switching. In using the SμKI mice to prepare hybridomas, the percentage of IgE-secreting hybridomas and the ratio of IgE to IgG hybridoma numbers increased when compared to results using the wild type mice.

Prior to our invention, there has not been a scientific paper or patent disclosure that describes the preparation of hybridomas by the conventional procedure of fusing mouse spleen cells with mouse myeloma cells and such hybridomas secrete human or “humanized” IgE that is specific to a defined protein component. Rare IgE-expressing B lymphocytes in human peripheral blood mononuclear cells and the low cell fusion efficiency of human B lymphocytes with human myelomas or lymphoma cell lines have hindered the preparation of hybridomas secreting human IgE. The Hakamata's group prepared a mite extract-specific human IgE hybridoma by using in vitro cytokine-activated and mite-extract-treated lymphocytes isolated from healthy donors (Hakamata 2000). The produced IgE mAb reacts with the mite extract rather than with a defined protein component (Hakamata 2000). In addition, a hybridoma secreting Der p 2-specific chimeric or “humanized” IgE was prepared by a gene transfection procedure (Aalberse 1996). In this study, a recombinant gene containing DNA segments encoding mouse heavy chain variable region specific for Der p 2 joined with human ε constant region and a geneticin-resistant protein was transfected into a mouse Der p 2-specific hybridoma variant, which had already lost its γ2b heavy chain gene. After drug selection of transfected cells and reactivity tests for survival clones, the humanized IgE hybridoma specific to Der p 2 was prepared (Aalberse 1996).

### SUMMARY OF THE INVENTION

Transgenic non-human animals are disclosed which are capable of producing abundant polyclonal “humanized” IgE. In this invention disclosure, “humanized” IgE represents that the constant region of the immunoglobulin ε of the IgE, encompassing CH1, CH2, CH3, CH4, M1, and M2, is human and variable region is the animal's own. M1 and M2, which are respectively encoded by two “membrane exons” in the ε gene, represent two contiguous peptide segments that form the membrane-anchor peptide of 69 amino acid residues extending from the C-terminal of membrane-bound ε heavy chain (mε). In some embodiments, the humanized IgE also include a form of IgE, in which the constant regions of both ε heavy chain and κ light chain are human and the variable regions of the heavy and light chains are the animal's own. The transgenic animals are mouse, rat, and rabbit, for which methods for genetic manipulation and alteration are established. Thus, for these transgenic animals, the coding sequences of CH1, CH2, CH3, M1, and M2 for one of the Cγ immunoglobulin gene are replaced by the corresponding coding sequences of human Cε immunoglobulin gene. It is noted that a γ chain has only 3 CH domains and also has a C-terminal membrane anchor peptide that is encoded by two membrane exons.

A preferred embodiment of this invention is mouse and the Cγ gene chosen is Cγ1. For further enhancing the “humanness” antigenic property of the humanized IgE, the transgenic mouse strain is crossed with a transgenic mouse strain, in whose genome the coding region of the constant region of the mouse κ chain is replaced by the corresponding coding segment of human κ chain, to obtain the homozygous transgenic mouse strain that harbor human Cε and Cκ constant region genes.

The invention also pertains to the applications of the transgenic animals constructed as described above in producing serum containing humanized IgE, antigen-specific humanized IgE, and hybridomas producing antigen-specific humanized IgE. For preparing antiserum containing antigen-specific IgE and for preparing hybridomas secreting antigen-specific humanized IgE in transgenic mice or rats, the animals are immunized with the specified antigens, such as dust mites of particular strain or region, pollens of a particular tree or grass, shed dander of cats, or isolated antigens of certain foods, to boost the proportion of antigen-specific humanized IgE in total IgE. The serum containing polyclonal humanized IgE, antisera containing antigen-specific humanized IgE, or the antigen-specific humanized monoclonal IgE can be applied for various immunoassays for measuring IgE or antigen-specific IgE in the sera of patients with IgE-mediated allergy.

### DETAILED DESCRIPTION OF THE INVENTION

### 1. Altering the Relative Abundance of Immunoglobulin Isotypes

The immunoglobulin heavy chain gene locus (IGHC) contains in one cluster of the genes encoding the constant regions of all of the classes and subclasses of heavy chains, including μ chain of IgM, δ chain of IgD, and γ chain of IgG, and α chain of IgA, and ε chain of IgE. In both human and mouse, the γ class has four subclasses and the α class has two subclasses. In human genome, the IGHC is arranged in the order of μ-δ-γβ-γ1-α1-γ2-γ4-ε-α2, and in the mouse genome, IGHC is arranged in the order μ-δ-γβ-γ1-γ2b-γ2a (or γ2c)-ε-α. The gene elements encoding each of the subclasses is separated from the neighboring subclass by the switch (S) regions involved in class switch recombination (CSR).

The immune-competent resting B lymphocytes bear surface membrane-bound IgM and IgD (mIgM and mIgD). Upon initial antigen stimulation, the first antibodies produced by the lymphocytes are of the IgM class. With continual or repeated antigen stimulation, the activated B lymphocytes expand, differentiate, and secrete antibodies toward the antigens. One important aspect of this antibody response is that the B cells undergo isotype-switching from originally IgM production to the production of another isotype. The regulation and the determination of isotypes are mediated by a network of cytokines, chemokines, transcription activators, and negative regulators. Following antigen stimulation, signaling pathways recruit those factors which regulate the expression of germ line transcripts and the switch regions of the individual genes (Chaudhuri and Alt 2004; Stavnezer and Amemiya 2004; Pan-Hammarstroem et al. 2007). CSR that effectuates the change in antibody class is a deletional recombination where the constant region gene of the heavy chain Cμ is replaced by a downstream C_{H} gene and the intervening sequences are excised as circular DNA. CSR is initiated by activation-induced deaminase acting within the S region, which is followed with double strand breaks, DNA damage response/repair pathways and nonhomologous end joining (Chaudhuri and Alt 2004). The Ig of different class and subclass is expressed at different levels. In general, IgG, IgA, and IgM are expressed at much higher levels than IgD and IgE. And between IgD and IgE, the latter is still much lower. In addition to the different levels of production among the different classes, the turnover rate of free Ig and the stabilization of each Ig class by its receptor contribute to the overall turnover kinetics, the abundance, and half-life of the Ig class.

The present invention pertains to genetically altering an animal, so that the IgE in the altered animal becomes humanized IgE and its production is much higher than the IgE in an unaltered animal host. For achieving this, a mouse, rat, or rabbit is used, because genetic alteration of the antibody genes in these animals can be achieved with existing tools of molecular biology and embryonic stem cell manipulation, and the information concerning the immunoglobulin gene complexes in these animals. Furthermore, among these animals, mouse is a good choice because the time for reproduction is short and the tools for preparing transgenic strains are well established.

To increase the overall IgE levels, the coding sequences for the constant region of one of Cγ immunoglobulin, such as Cγ1, which is expressed at high levels, is replaced by the coding sequence for the constant region of human Cε. In doing so, the regulatory sequences in the promoter and the S regions of the mouse own Cγ gene are kept, so that the control of expression of the knock-in human Cε may also achieve high expression. It is noted that since human IgE is not recognized by mouse FcεRI, the transgenic mice should not have adverse conditions even they produce large quantities of humanized IgE.

### 2. Construction of a Chimeric Transgene Comprising Human a Coding Sequences Replacing the Mouse Cγ1 Coding Sequences in Mouse Immunoglobulin Heavy Chain γ Gene Locus (mIGHG)

The replacement is achieved via homologous recombination between a designed construct and a mouse BAC clone containing the mouse IGHG locus (Clone ID RP24-258E20, FIG. 1A). The construct can be generated by PCR amplification incorporating the coding regions of human Cε CH1-CH2-CH3-CH4-M1-M2, flanked at either end with 2,000 bp each of the mouse sequences upstream and downstream, respectively, of the mouse Cγ1 gene at the recombination sites. The homologous recombination can be performed in E. coli using the Red®/ET® Recombination methodology (Gene Bridges GmbH, Dresden, Germany). Specifically, the homologous recombination occurs in two steps. First, a counter selection marker rpsL-neo replaces the mouse Cγ1 coding region for CH1-H—CH2-CH3-M1-M2 and is incorporated between the mouse homologous arms (the 2,000 bp sequences described above). “H” represents the hinge region. Then, the counter selection marker is replaced with the human Cε region encoding CH1-CH2-CH3-CH4-M1-M2.

### 3. Construction of a Chimeric Transgene Comprising Human Cκ Coding Sequences Replacing the Mouse Cκ Coding Sequences in Mouse Immunoglobulin Light Chain κ Locus (IGKC)

A construct is designed with PCR amplification incorporating human Cκ coding sequences flanked at either end with 50 bp each of the mouse sequences in the noncoding region upstream and downstream, respectively, of the mouse Cκ gene at the recombination sites. The construct is then integrated into a mouse BAC clone containing the IGKC locus (Clone ID RPCI23-5905, FIG. 1A) via Red®/ET® Recombination methodology in E. coli (Gene Bridges GmbH, Dresden, Germany). Again, the homologous recombination occurs in two steps. First, a counter selection marker rpsL-neo replaces the mouse Cκ coding region and is incorporated between the mouse homologous arms (the 50 bp sequences described above). Then, the counter selection marker is replaced with the human Cκ coding sequences.

### 4. Generation of Transgenic Mice Harboring the Chimeric Transgenes

The method for transgene transfer employs the embryonic stem cell (ES). ES cells are obtained from pre-implantation embryos cultured in vitro and fused with embryos. Transgenes can be efficiently introduced into the ES cells by electroporation, retrovirus-mediated transduction or other methods. The preferred method is electroporation. Such transformed ES cells can thereafter be combined with blastocysts from a nonhuman animal. The ES cells thereafter colonize the embryo and contribute to the germ line of the resulting chimeric animal.

Homologous recombination can also be used to introduce transgenes. Homologous recombination can be mediated by either RecE/RecT (RecE/T) or Red α/β. In E. coli, any intact, independently replicating, circular DNA molecule can be altered by RecE/T or Red α/β mediated homologous recombination with a linear DNA fragment flanked by short regions of DNA sequence identical to regions present in the circular molecule. Integration of the linear DNA fragment into the circular molecule by homologous recombination replaces sequences between its flanking sequences and the corresponding sequences in the circular DNA molecule.

The homologous recombination described in sections 3 and 4 above yield transgenes comprising modified mouse BAC clones harboring the human Cε coding sequences and Cκ coding sequences, respectively. Each transgene is then introduced via electroporation into embryonic stem cells of mouse strain C57BL/6 where homologous recombination of the transgene and the corresponding endogenous gene locus takes place. The colonies verified to contain successfully recombined transgenes are then injected into blastocysts of C57BL/6, which are subsequently transferred into the uterus of pseudopregnant mice of the C57BL/6J-c2J strain. The embryos are allowed to develop into chimeric mice, which are then monitored to produce transgenic mice as in the standard procedures listed above.

The transgenic mice harboring the human Cε coding region substituting mouse Cγ1 coding region and those harboring the human Cκ coding region substituting mouse Cκ coding region are then crossed to produce mice harboring both transgenes in place of the respective endogenous coding sequences. The resulted mouse strain that harbors both transgenes is used for the production of antigen-specific humanized IgE and hybridomas secreting antigen-specific humanized IgE.

### 5. Production of Antiserum Containing Antigen-Specific Humanized IgE and Hybridomas Secreting Antigen-Specific Humanized IgE

The transgenic mice resulted from the crosses as described in section 4 are used to generate antigen-specific humanized IgE and hybridomas secreting antigen-specific humanized IgE. Two examples of specific IgE production are: (i) antigens, such as dust mites, and weed, grass or tree pollens, and (ii) Geohelminth parasites, such as Necator americanus (human hookworm) and Trichuris suis (pig whipworm).

### Examples

### 1. Preparation of Recombination-Potent Bacterial Artificial Chromosome (BAC)-Bearing Bacteria and Replacing Mouse Cγ1-Encoding Gene with a Prokaryotic Selection DNA Cassette

The bacterial clone carrying BAC RP24-258E20, which contains gene exons encoding mouse four Cγ heavy chains (FIG. 1A and FIG. 2, sequence a), was purchased from BACPAC Resources Center. The gene replacement was accomplished by using the Red/ET-based recombination system.

To prepare recombination-potent BAC-bearing bacteria, the pRed/ET plasmid DNA which encodes enzymatic proteins essential for mediating homologous recombination was delivered into the BAC-bearing bacteria. A single colony of BAC-bearing bacteria grown on LB agar with chloramphenicol and streptomycin was inoculated in 1 ml LB medium with antibiotics. After culturing at 37° C. overnight, the bacteria (30 μl) were added into 1.4 ml of LB medium with antibiotics and cultured at 37° C. for 2 hours. The bacteria were placed on ice followed by centrifugation at 11,000 rpm for 30 s and the supernatant was removed. The pellet was washed with 1 ml of chilled 10% glycerol and centrifuged to remove the supernatant. The pellet was resuspended in 20-30 μl of chilled 10% glycerol and placed on ice. The pRed/ET plasmid DNA (20 ng) was added into the bacteria and mixed briefly. The mixture was transferred into a chilled 1-mm electroporation cuvette and shocked at 1.8 kV, 200 ohms, and 25 μF for 4.5˜5.0 ms. The electroporation condition was used in the following examples. LB medium (1 ml) was added to resuspend the bacteria and then transferred into a culture vessel. The bacteria were cultured at 30° C. for 70 mins and 100 μl of cultured bacteria was spread onto an LB agar plate with chloramphenicol and tetracycline. The plate was incubated at 30° C. overnight for growth of pRed/ET plasmid DNA-carrying bacteria which were recombination-potent.

The mouse Cγ1-encoding gene in the recombination-potent BAC-bearing bacteria was replaced by a prokaryotic selection DNA cassette which contains a hybrid rpsL-neo gene that confers streptomycin-sensitive and kanamycin-resistant selection for transfected bacteria. A single colony of the recombination-potent BAC-bearing bacteria was inoculated in 1 ml of LB with chloramphenicol and tetracycline. After culturing at 30° C. overnight, 30 μl of cultured bacteria were added into 1.4 ml of LB medium with antibiotics followed by culturing at 30° C. for 2 hours. L-arabinose at final 10% was added into the culture bacteria with culturing at 37° C. for another 1 hour. The bacteria were placed on ice and then centrifuged at 11,000 rpm for 30 s to remove the supernatant. The pellet was then washed with 1 ml of chilled 10% glycerol and centrifuged to remove the supernatant. The pellet was then resuspended in 20-30 μl of chilled 10% glycerol and placed on ice. The DNA stretch containing the hybrid rpsL-neo gene flanked with two 50-bp DNA sequences corresponding to intronic sequences of the overhangs of mouse Cγ1-encoding gene (SEQ ID NO:1) was prepared by polymerase chain reaction (PCR) with specific primers (TABLE 1, primers G1_CH1-rpsL-neo+ and G1_M2-rpsL-neo−). The purified DNA product (100-200 ng) was added into the resuspended bacteria with brief mix. The mixture was transferred into a chilled 1 mm cuvette for electroporation. LB medium (1 ml) without antibiotics was added to resuspend the shocked bacteria and transferred into a culture vessel. The bacteria were cultured at 37° C. for 70 mins and 100 μl of the cultured medium was spread onto an LB agar plate containing chloramphenicol, kanamycin, and tetracycline. The plate was incubated at 30° C. overnight and the grown colonies were screened for identifying bacteria carrying rpsL-neo knock-in BAC by colony PCR with specific primers (TABLE 2, primers G1_CH1-up-sc+ and rpsL_sc−). Identified clones were grown onto an LB agar plate with antibiotics at 30° C. overnight.

TABLE 1

| | PCR product | |
|---|---|---|
| Primers for cloning | lengths (bp) | Sequence ID |
| For human Cε transgene | | |
| G1_CH1-rpsL-neo+ | 4084 | SEQ ID NO: 8 |
| Gl_M2-rpsL-neo− | | SEQ ID NO: 9 |
| EcoR-mIGHG1-2kInt+ | 2012 | SEQ ID NO: 10 |
| Cla-mIGHG1-CH1Int− | | SEQ ID NO: 11 |
| Cla-hIGHE-CH1Ex+ | 4080 | SEQ ID NO: 12 |
| hIGHE_me2Int− | | SEQ ID NO: 13 |
| mIgG1Int + hIGHEM2-Cla-del+ | 8797 | SEQ ID NO: 14 |
| mIgG1Int + hIGHEM2-Cla-del− | | SEQ ID NO: 15 |
| Sac_mIGHG1m2-Int+ | 2254 | SEQ ID NO: 16 |
| Xho_mIGHG1polyA− | | SEQ ID NO: 17 |
| G1_M2_5h-neo+ | 1716 | SEQ ID NO: 18 |
| G1_M2_5h-neo− | | SEQ ID NO: 19 |
| For mouse Cκ transgene | | |
| mIGKC-rpsL-neo+ | 1419 | SEQ ID NO: 20 |
| mIGKC-rpsL-neo− | | SEQ ID NO: 21 |
| mIGKChm-hIGKC+ | 424 | SEQ ID NO: 22 |
| mIGKChm-hIGKC− | | SEQ ID NO: 23 |
| mIGKCInt5hT71oxP+ | 1716 | SEQ ID NO: 24 |
| mIGKCInt5hSP61oxP− | | SEQ ID NO: 25 |

TABLE 2

| | PCR product | |
|---|---|---|
| Primers for screening | lengths (bp) | Sequence ID |
| For human Cε transgene | | |
| G1_CH1-up-sc+ | 661 | SEQ ID NO: 26 |
| rpsL_sc− | | SEQ ID NO: 27 |
| G1_CH1up-sc+ | 478 | SEQ ID NO: 28 |
| hIGHE-CH1− | | SEQ ID NO: 29 |
| G1_M2pA2k-sc+ | 441 | SEQ ID NO: 30 |
| pgk_neo− | | SEQ ID NO: 31 |
| p1 | 257 | SEQ ID NO: 36 |
| p2 | | SEQ ID NO: 37 |
| p1 | 362 | |
| p3 | | SEQ ID NO: 38 |
| For mouse Cκ transgene | | |
| m-hIGKC-sep+ | 300 | SEQ ID NO: 32 |
| mIGKC-Int1− | | SEQ ID NO: 33 |
| mIGKC-Int+ | 625 | SEQ ID NO: 34 |
| rpsL_sc− | | |
| mIGKC-neo+ | 471 | SEQ ID NO: 35 |
| pgk_neo− | | |
| p4 | 207 | SEQ ID NO: 39 |
| p6 | | |
| p5 | 300 | SEQ ID NO: 40 |
| p6 | | SEQ ID NO: 41 |

### 2. Construction of the DNA Stretch with Human Cε-Encoding Gene for Recombination and the Human Cc-Encoding Gene Knock-in BAC

The DNA stretch containing the human Cε-encoding gene flanked with 5′ and 3′ overhang sequences of the mouse Cγ1-encoding gene (SEQ ID NO:2) was prepared by PCR and DNA cloning techniques. The steps to construct the DNA stretch were shown in FIG. 1B. Primers with restriction enzyme sites for amplifying individual 5′ and 3′ overhangs of the mouse Cγ1 and the human Cε-encoding gene were listed in Table 1. The BAC RP24-258E20 was used as DNA templates for amplifying the 5′ and 3′ overhangs of the mouse Cγ1 with primers EcoR-mIGHG1-2kInt+/Cla-mIGHG1-CH1Int− and Sac_mIGHG1m2-Int+/Xho_mIGHG1polyA− (TABLE 1), respectively. The genomic DNA isolated from SKO-007, a human IgE myeloma cell line, was served as a template for amplifying the human Cε-encoding gene by PCR with primers Cla-hIGHE-CH1Ex+ and hIGHE_me2Int− (TABLE 1). Each amplified DNA fragment was ligated into a TA vector (Real Biotech Corporation, Taiwan) for sequence verification and plasmid DNA preparation. In brief, the DNA fragment of 5′ overhang purified from the plasmid DNA digested with EcoRI and ClaI restriction enzymes (New England Biolabs, MA) was ligated with the human Cε gene plasmid DNA digested with the same restriction enzymes. The ClaI− reacting sequence in the resultant plasmid DNA was further eliminated by using overlapped primers without incorporating the ClaI-reacting sequence in each direction primer to amplify the plasmid DNA by PCR with primers mIgG1Int+hIGHEM2-Cla-del+ and mIgGlInt+hIGHEM2-Cla-del− (TABLE 1). The amplified linear DNA fragment was delivered into a transformation-competent bacterial host to produce a circular plasmid DNA. The DNA fragment of the human CE-encoding gene with 5′ overhang was prepared by digesting the circular plasmid DNA with EcoRI and SalII restriction enzymes (New England Biolabs), and was ligated into the 3′ overhang plasmid DNA digested with the same enzymes. The DNA stretch of human Cε-encoding gene with overhangs was prepared by digesting the ligated plasmid DNA with EcoRI and XhoI restriction enzymes (New England Biolabs). The SalII, EcoRI, and XhoI-reacting sequences are present in genomic sequences of the human Cε gene and the mouse Cγ1 overhangs.

The rpsL-neo gene in the knock-in BAC was further replaced by the human Cε-encoding gene. A single colony of bacteria bearing rpsL-neo gene knock-in BAC was inoculated in 1 ml LB medium with chloramphenicol, kanamycin, and tetracycline. After culturing at 30° C. overnight, 30111 of cultured bacteria were added into 1.4 ml of LB medium with antibiotics followed by culturing at 30° C. for 2 hours. L-arabinose at final 10% was added into the bacteria with growing at 37° C. for another 1 hour. The bacteria were then placed on ice followed by centrifugation at 11,000 rpm for 30 s to remove the supernatant. The pellet was washed with 1 ml of chilled 10% glycerol and centrifuged again to remove the supernatant. The pellet was resuspended in 20-30 μl of chilled 10% glycerol and placed on ice. The purified human Cε DNA stretch (100-200 ng) was added into the resuspended bacteria with brief mix. The mixture was transferred into a chilled 1 mm cuvette for electroporation. LB medium (1 mL) was then added to resuspend the shocked bacteria followed by transferring to a culture vessel. The bacteria were cultured at 37° C. for 70 mins and 100 μl of the cultured bacteria were spread onto an LB agar plate containing chloramphenicol and streptomycin. The plate was incubated at 30° C. overnight and the grown colonies were screened for identifying the bacteria carrying the human Cε gene knock-in BAC (FIG. 2, sequence b) by PCR with specific primers (TABLE 2, primers G1_CH1up-sc+ and hIGHE-CH1−). Identified clones were streaked onto a LB agar plate with antibiotics and grown at 30° C. overnight.

### 3. Construction of the Neo-Inserted Human Cε Gene Knock-in BAC for Gene Targeting in ES Cells

The prokaryotic/eukaryotic neo-expressing cassette (SEQ ID NO:3) was inserted into the 3′ overhang of the mouse Cγ1-encoding gene for selection of neomycin-resistant human Cε gene-knocked-in ES cells. The DNA stretch of the cassette flanked by 50-bp DNA sequences in the 3′ overhang of the mouse Cγ1-encoding gene was prepared by PCR with specific primers (TABLE 1, primers G1_M2_5 h-neo+ and G1_M2_5 h-neo−). A single colony of bacteria bearing human Cε-encoding gene knock-in BAC was inoculated in 1 ml LB medium with chloramphenicol and streptomycin for culturing at 30° C. overnight. The cultured bacteria (30 μl) were added into 1.4 ml LB medium with antibiotics and continuously cultured at 30° C. for 2 hours. L-arabinose at final 10% was added into the bacteria with culturing at 37° C. for another 1 hour. The cultured bacteria were placed on ice followed by centrifugation at 11,000 rpm for 30 s to remove the supernatant. The pellet was washed with 1 ml of chilled 10% glycerol and centrifuged again to remove the supernatant. The pellet was resuspended in 20-30 μl of chilled 10% glycerol and placed on ice. The purified PCR product (100-200 ng) was added into the resuspended cell pellet with brief mix. The mixture was transferred into a chilled 1 mm cuvette for electroporation. LB medium (1 mL) was added to resuspend the shocked bacteria followed by transferring into a culture vessel. The bacteria were cultured at 37° C. for 70 mins and 100 μl of the cultured bacteria were spread onto a LB agar plate containing chloramphenicol and kanamycin. The plate was incubated at 37° C. overnight and the grown colonies were screened for identifying bacteria carrying the neo-inserted BAC (FIG. 2, sequence c) by PCR with specific primers (TABLE 2, primers G1_M2pA2k-sc+ and pgk_neo−). The identified bacteria were further amplified to isolate gene knock-in BAC DNA for transfection of ES cells

### 4. Construction of the Neo-Inserted Human κ Chain Exon Knock-in BAC

The BAC DNA RP23-5905 which contains the mouse κ chain-encoding exon (FIG. 1A and FIG. 3, sequence d) was purchase from BACPAC Resources Center. The procedures of gene replacement were followed by using the Red/ET-base recombination system. The mouse κ chain exon was first replaced by the rpsL-neo-expressing cassette (SEQ ID NO:4). The bacteria bearing BAC RP23-5905 were prepared to carrying the pRed/ET plasmid DNA by procedures described in Example 1 and used for electroporation. The DNA stretch of the rpsL-neo-expressing cassette flanked with two 50-bp DNA sequences corresponding to intronic sequences flanking the mouse κ chain exon was prepared by PCR with specific primers (TABLE 1, primers mIGKC-rpsL-neo+ and mIGKC-rpsL-neo−). The purified PCR product of rpsL-neo-expressing cassette (100-200 ng) was added into the bacteria followed by electroporation. LB medium (1 mL) was added to resuspend the shocked bacteria and transferred into a culture vessel. The bacteria were cultured at 37° C. for 70 mins and 100 μl of the cultured bacteria were spread onto a LB agar plate containing chloramphenicol, kanamycin, and tetracycline. The plate was incubated at 30° C. overnight and the grown colonies were screened for identifying bacteria carrying rpsL-neo knock-in BAC by PCR with specific primers (TABLE 2, primers m-hIGKC-sep+ and mIGKC-Intl−). The identified bacteria were cultured in LB medium with antibiotics at 30° C. overnight for the use in the following step.

The DNA stretch of the human Cκ chain exon flanked with two 50-bp DNA stretches corresponding to intronic sequences flanking the mouse Cκ chain exon (SEQ ID NO:5) was prepared by PCR with specific primers (TABLE 1, primers mIGKChm-hIGKC+ and mIGKChm-hIGKC−). A human genomic DNA isolated from a healthy donor's blood was used as the DNA template for amplifying the human Cκ chain exon in PCR. The cultured bacteria with rpsL-neo knock-in BAC were prepared for electroporation with the purified PCR product (100-200 ng) of human Cκ chain exon. LB medium (1 mL) was added to resuspend the shocked bacteria and transferred into a culture vessel. The bacteria were cultured at 37° C. for 70 mins and 100 μl of the cultured bacteria were spread onto a LB agar plate containing chloramphenicol, streptomycin. The plate was incubated at 30° C. overnight and the grown colonies were screened for identifying the bacteria carrying the human Cκ chain exon knock-in BAC (FIG. 3, sequence e) by PCR with specific primers (TABLE 2, primers mIGKC-Int+ and rpsL_sc−). The identified bacteria were cultured in LB medium with antibiotics at 30° C. overnight for the use in the following step.

The DNA stretch of the loxP-flanked neo-expressing cassette flanked with two 50-bp DNA sequences corresponding to intronic sequences of 3′ overhang of the mouse Cκ chain exon (SEQ ID NO:6) was prepared by PCR with specific primers (TABLE 1, primers mIGKCInt5hT7loxP+ and mIGKCInt5hSP6loxP−). The cultured bacteria with the human Cκ chain exon knock-in BAC were prepared for electroporation with the purified PCR product (100-200 ng) of the neo− expressing cassette. LB medium (1 mL) was added to resuspend the shocked bacteria and transferred into a culture vessel. The bacteria were cultured at 37° C. for 70 mins and 100 μl of the cultured bacteria were spread onto an agar plate containing chloramphenicol and kanamycin. The plate was incubated at 37° C. overnight and the grown colonies were screened for identifying the bacteria carrying the neo-inserted human Cκ chain exon knock-in BAC (FIG. 3, sequence f) by PCR with specific primers (TABLE 2, primers mIGKC-neo+ and pgk_neo−). The identified bacteria were further amplified to isolate gene knock-in BAC DNA for transfection of ES cells.

### 5. Generation and Genotyping of Transgenic Mice

The preparation of gene knock-in ES cells and implantation of ES cells into pseudo-pregnant female mice were followed with standard techniques. In brief, the knock-in BAC DNA was linearized by NruI and NotI restriction enzyme digestion (New England Biolabs) and delivered into ES cells derived from C57BL/6 mice by electroporation followed by culturing in the geneticin-containing medium. After drug selection, each resistant ES cell clone was verified with PCR to obtain the cells with DNA replacement at the correct site of the target gene. The gene knock-in ES cells were transferred to the blastocysts and then implanted into the pseudo-pregnant C57BL/6J-c2J mice (The Jackson Laboratory, ME). The offspring were bred and mated to generate mice with two homozygous alleles of the transgene (the human Cε gene and the human Cκ gene, respectively). Mice carrying the homozygous knock-in allele were further mated with B6.FVB-Tg(Ella-cre)C5379Lmgd/J mice (The Jackson Laboratory) to remove the loxP-flanked neomycin cassette. The human Cε gene knock-in (hCε^{+/+}) and the human Cκ gene knock-in (hCκ^{+/+}) mice were further cross-mated to generate humanized IgE mice which harbored double homozygous alleles of the two genes (hCε^{+/+}hCκ^{+/+}) and were denoted as HεκKI mice. The mouse littermates harboring different allelic combinations, such as hCε^{-/-} hCκ^{+/+}, hCε^{+/-}hCκ^{+/+}, and hCε^{+/+}hCκ^{+/+}, were obtained by inbred mating of mice bearing hCε^{+/-} hCκ^{+/+}.

To characterize the genotypes of the heavy chain or the light chain transgenic mice, the genomic DNA was purified from a piece of mouse tail tissue with an EasyPure Genomic DNA mini kit (Bioman Scientific, Taiwan) and with the procedure provided in the manual. The purified DNA was used in PCR with primers p1, p2 and p3 for hCε knock-in mice (FIG. 4A and TABLE 2) and p4, p5 and p6 for hCκ knock-in mice (FIG. 4B and TABLE 2). The amplified DNA sizes with each primer pair were shown in Table 2. The genotypes of the heavy chain transgene with homozygous hCε⁺/+, heterozygous hCε^{+/-}mCγ1^{+/-}, or wild type mCγ1^{+/+}, denoted as hCε/hCε, hCε/mCγ1, and mCγ1/mCγ1 in FIG. 4C, respectively, were revealed on an agarose gel by DNA electrophoresis (FIG. 4C). The genotypes of the light chain transgene with homozygous hCκ^{+/+}, heterozygous hCκ^{+/-}mCκ^{+/-}, or wild type mCκ^{+/+}, denoted as hCκ/hCκ, hCκ/mCκ, and mCκ/mCκ in FIG. 4C, respectively, were also shown on the same DNA agarose gel (FIG. 4C).

The genomic DNA of the heavy chain transgenic mice was further verified by Southern blotting analyses. Five microgram of genomic DNA was digested overnight by BamHI restriction enzymes (New England Biolabs). The digested genomic DNAs were loaded into a 0.8% agarose gel and electrophoresed at 50 V for 1.5 hours followed by submerging the gel in denaturation solutions (0.5M NaOH and 1.5M NaCl) for 15 mins twice with gentle shaking. The gel was rinsed with distilled water and submerged in neutralization solutions (0.5M Tris-HCl, pH 7.5 and 1.5M NaCl) for 15 mins twice with gentle shaking followed by equilibrating the gel in 20×SSC solutions (3 M sodium chloride and 300 mM trisodium citrate) over 10 mins. A piece of Whatman® 3MM paper (Sigma-Aldrich) soaked with 20×SSC solutions was placed in a reservoir filled with 20×SSC solutions. The gel was transferred onto the Whatman® 3MM paper followed by topping with a piece of nylon membrane (Roche Diagnostics GmbH, Germany). A piece of Whatman® 3MM paper rinsed with 2×SSC solutions was placed onto the membrane, and a stack of tissue paper was then transferred onto the Whatman® 3MM paper with a weight on the top. After transferring for 16-24 hrs, the membrane was baked in an oven at 80° C. for 2 hr for the following use.

The digoxigenin (dig)-labelled hybridization probe (FIG. 4A and SEQ ID NO:7) was prepared by GoTaq Flexi DNA polymerase (Promega, WI) and DIG DNA Labeling Mix (Roche) in PCR with the primer pair mg1probe+/mg1probe− (TABLE 3). The PCR product containing dig-labelled probe (2 μl) was diluted in 50 μl of sterile distilled water in a 2-ml tube followed by boiling at 100° C. for 5 mins. The tube was chilled on ice immediately, and 1.75 ml of DIG Easy Hyb hybridization buffers (Roche) were added into the tube. After mixing, the solution was incubated with the membrane in a bag. The hybridization was carried out by placing the bag in an oven at 65° C. for 16-24 hrs. The membrane was washed twice with 2×SSC solutions containing 0.1% sodium dodecyl sulfate (SDS, Sigma-Aldrich) and twice with warm (65° C.) 0.5×SSC solutions containing 0.1% SDS for 15 mins with gentle shaking. After cooling down the membrane to room temperature, the membrane was washed and blocked with buffers in a DIG Wash and Block Buffer Set (Roche). Anti-DIG-AP Fab fragments (Roche) were 10,000-fold diluted in blocking buffers and incubated with the membrane for 30 mins. After washing twice with washing buffers, the membrane was equilibrated with detection buffers in a DIG Wash and Block Buffer Set (Roche) for 3 mins with gentle shaking. After removing detection buffers, the membrane was incubated with 0.5 ml of CDP-star chemiluminescent substrate (Roche) for 5 mins, and luminescence signals were detected with a LAS-3000 Imaging system (Fujifilm, Japan). The results showed that the probe yielded a 1.2-kb band for the WT allele versus a 3.7-kb band for the human Cε knock-in allele (FIG. 4D).

TABLE 3

| | PCR product | |
|---|---|---|
| Primers for detection | lengths (bp) | Sequence ID |
| For Southern blotting | | |
| mg1probe+ | 937 | SEQ ID NO: 42 |
| mg1probe− | | SEQ ID NO: 43 |
| For real-time qPCR | | |
| RQ-Cg1+ | 264 | SEQ ID NO: 44 |
| RQ-Cg1− | | SEQ ID NO: 45 |
| RQ-Ce+ | 125 | SEQ ID NO: 46 |
| RQ-Ce− | | SEQ ID NO: 47 |
| RQ-BA+ | 300 | SEQ ID NO: 48 |
| RQ-BA− | | SEQ ID NO: 49 |

### 6. Real-Time RT-PCR for Detecting Human ε mRNA in the Spleens of Transgenic Mice

Total RNA of spleen cells from three transgenic mice hCε/hCε, hCε/mCγ1, and mCγ1/mCγ1 bearing the human Cκ gene, respectively, was prepared by using a PureLink RNA Mini Kit (Life Technologies, CA). The purified total RNA (5 μg) was used for cDNA preparation with a Superscript III reverse transcriptase kit (Life Technologies). The cDNA (100 ng) was used in each reaction of quantitative PCR (qPCR) with SYBR® Green PCR Master Mix (Applied Biosystems, CA). Reactions were carried out and signals were analyzed with StepOnePlus™ Real-Time PCR Systems (Applied Biosystems). Primer pairs for amplifying the constant regions of the mouse IgG1 (RQ-Cg1+/RQ-Cg1−) and human IgE (RQ-Ce+/RQ-Ce−) as well as mouse beta-actin (RQ-BA+/RQ-BA−) were listed in Table 3. The SYBR® Green signals for quantifying the amount of amplified DNA products of mouse IgG1 and human IgE were normalized with the signals of mouse beta-actin in the parallel reactions. Triplicated qPCR reactions were run for each mouse cDNA and three mouse spleens were studied for each genotype. Results showed that mouse γ1 mRNA was undetectable in hCε/hCε mice (FIG. 5A) and mCγ1/mCγ1 mice did not express human ε mRNA (FIG. 5B). The expression amount of human ε mRNA in hCε/hCε mice was 1.8 folds as much as that in hCε/mCγ1 mice (FIG. 5B), and the expression amount of mouse γ1 mRNA in mCγ1/mCγ1 mice was 2.1 folds as much as that in hCε/mCγ1 mice (FIG. 5A).

### 7. ELISPOT for Detecting Humanized IgE-Secreting B Cells in the Spleens of Transgenic Mice

Three mice (7-8 weeks old) in each group of the 3 genotypes (hCε/hCε, hCε/mCγ1, and mCγ1/mCγ1) were immunized subcutaneously three times with 50 μg of papain (Sigma-Aldrich, MO) emulsified with TiterMax® Gold (Sigma-Aldrich) at day 1, day 22 and day 36. The mice were sacrificed at days 50, 52 and 54 for three independent experiments and the single splenocytes were prepared by grinding spleens with frosted glass slides. The splenocytes were washed with RPMI medium (Life Technologies) twice and resuspended in RPMI medium plus 10% fatal bovine serum (FBS) and penicillin-streptomycin (Life Technologies). For preparing micro-well plates for ELISPOT analyses, MultiScreenHTS plates (Millipore, Mass.) were socked with 15 μl of 35% ethanol for 1 min and washed with phosphate buffered saline (PBS) three times followed by coating with 1 μg per well of polyclonal goat anti-mouse IgG1 (Southern Biotech), goat anti-mouse IgG-Fc (Bethyl Laboratories, TX), goat anti-mouse IgE (Bethyl Laboratories), or goat anti-human IgE antibodies (Bethyl Laboratories) in 100 μl PBS at 4° C. overnight. The plates were washed with PBS three times and blocked with 200 μl of RPMI medium plus 10% FBS at 37° C. for 1 hr. After washing plates with PBS three times, 100 μl of cell suspension (5×10⁵ splenocytes) were dispensed into the individual wells. The splenocytes were cultured in an incubator at 37° C. for 16-24 hrs. The plates were washed with PBS plus 0.1% Tween 20 (Sigma-Aldrich) six times and blocked with 1% bovine serum albumin (BSA)/PBS for 1 hr. After washing with PBS three times, 100 III of horseradish peroxidase (HRP)-conjugated goat anti-mouse IgG1 (Southern Biotech), goat anti-mouse IgG-Fc (Bethyl Laboratories), goat anti-mouse IgE (Bethyl Laboratories), or goat anti-human IgE antibodies (Bethyl Laboratories) diluted 10,000 folds in 1% BSA/PBS were dispensed into each corresponding well. After incubation at room temperature for 2 hrs and washing with PBS 8 times, the wells each were added 100 μl of AEC solution (Life technologies) and incubated in the dark at room temperature for 30 minutes. After washing with distilled water 5 times, the wells were scanned, and spots were counted with an AID iSpot FluoroSpot Reader System (AID Diagnostika GmbH, Germany). The results showed that mouse IgG1-secreting B cells were undetectable in the spleens of hCε/hCε mice in which the number of total mouse total IgG-secreting B cells was comparable with that in hCε/mCγ1 and mCγ1/mCγ1 mice (FIG. 6A). In the spleens of hCε/hCε mice, the number of humanized IgE-secreting B cells was much lower than mouse IgG-secreting B cells (FIGS. 6A and 6B). Several humanized IgE-secreting B cells, as well as mouse IgE-secreting B cells, were detected in the three spleens of hCε/hCε or hCε/mCγ1 mice (FIG. 6B).

### 8. Measurement of Serum Titers of Different Ig Isotypes in Papain-Immunized Transgenic Mice

Papain is a protease and present in the latex of papaya tree. It is also an allergic component in latex-sensitive individuals. The effects of papain to stimulate IgE response in mice have been investigated. To study antibody response upon papain immunization in the three transgenic mice, the serum concentrations of different Ig isotypes were determined with ELISA in the example. Papain (Sigma-Aldrich) at the dose of 50 μg per mouse was emulsified with TiterMax® Gold Adjuvant (Sigma-Aldrich) and injected into the mice subcutaneously. The second injection was performed four weeks after the first injection and the blood was sampled at week 0 (pre-immune), week 2, week 4, and week 6. Concentrations of humanized IgE, mouse IgE, and mouse IgM were determined by using ELISA quantitation sets (Bethyl Laboratories) and the measurement procedures were followed according to the manuals. Concentrations of mouse IgG1, IgG2b, IgG2c, and IgG3 were detected by using polyclonal goat anti-Ig isotype-specific antibodies and polyclonal HRP-conjugated goat anti-Ig isotype-specific antibodies systems (SouthemBiotech). The mouse reference serum (Bethyl Laboratories) was used as the calibration standard for each mouse IgG1, IgG2b, IgG2c, and IgG3. The ELISA technique was followed by a standard procedure. In brief, polyclonal anti-Ig isotype-specific antibodies were diluted in the coating buffer (sodium bicarbonate, pH 9.6) and added into polystyrene wells. After incubation at 4° C. overnight, wells were washed with phosphate buffered saline (PBS) and blocked with 1% BSA/PBS. After incubation at room temperature for 1 hour, wells were washed with PBS three times and diluted mouse sera were added into wells for measuring concentrations of different Ig isotypes. Mouse sera were diluted in blocking buffers in 4 folds for human and mouse IgE measurement and in 4,000 folds for mouse IgM, IgG1, IgG2b, IgG2c and IgG3 measurement, respectively. After incubation for 2 hrs and washing with PBS three times, HRP-conjugated goat anti-Ig isotype-specific antibodies diluted in a proper concentration in blocking buffers were added into wells and incubated for 1 hr. After washing with PBS six times, the HRP substrate NeA-Blue (Clinical Science Products, MA) was added into wells for color development and colorimetric measurement with a Model 680 microplate reader (BioRad Laboratories, CA).

The results showed that serum levels of each Ig isotype increased in the three transgenic mice after papain immunization (FIG. 7). The IgG1 levels of immunized hCε/mCγ1 mice were comparable with that of immunized mCγ1/mCγ1 mice (FIG. 7). The humanized IgE levels of immunized hCε/mCγ1 mice were a half of that of immunized hCε/hCε mice (FIG. 7). In hCε/hCε mice, the serum levels of humanized IgE were about ten-fold higher than those of mouse IgE before or after papain immunization (FIG. 7).

### 9. Generation of Defined Protein Component-Specific Humanized IgE Hybridoma with the Splenocytes of Immunized HεκKI Mice

Papain, one of the allergic protein components in latex products, was used to prepare defined protein component-specific humanized IgE hybridomas. The papain-specific humanized monoclonal IgE was prepared by using a standard immunization procedure and a standard hybridoma technique. In brief, HεκKI mice of 7-8 weeks old were immunized with 50 μg of papain (Sigma-Aldrich) emulsified with Freund's complete adjuvant (Sigma-Aldrich) subcutaneously. After 3 weeks, the mice were injected with papain emulsified with Freund's incomplete adjuvant (Sigma-Aldrich) subcutaneously twice at a 2-week interval. The mice were then injected with 100 μg of papain intraperitoneally 3 days before sacrifice for hybridoma preparation. To prepare hybridomas, the spleen cells isolated from the immunized mouse were fused with mouse FO myeloma cells by using 50% (w/v) polyethylene glycol 1500 (Roche). The fused cells were then grown in hypoxanthine-aminopterin-thymidine selection medium for 10-12 days and the cultured supernatants of hybridomas were screening with ELISA to identify papain-specific humanized IgE hybridomas. To prepare ELISA, papain diluted in the coating buffer (10 μg/ml) was added into polystyrene wells and incubated at 37° C. for 1 hour. After washing with PBS and blocked with 1% BSA for 1 hour, the culture supernatants were added into wells and then placed at room temperature for 1 hour. After washing with PBS, HRP-conjugated goat anti-human IgE (1:10000 dilutions, Bethyl Laboratories) was added to wells and incubated at room temperature for 1 hour. After extensive washes, the HRP substrate was added into wells for color development and colorimetic measurement. Three papain-specific hybridomas producing the human ε constant region of the heavy chain, denoted as 1C6, 6D10, and 34C2, were identified (FIG. 9A). These three hybridomas also secreted mAbs with human κ constant region rather than the mouse λ constant region of the light chain (FIG. 9B).

To purify human or humanized IgE antibodies, a humanized IgG1 mAb, Omalizumab (Norvatis), specific for human IgE was coupled onto the CNBr-activated Sepharose 4 Fast Flow resin (GE Healthcare). The coupling procedures were followed according to the manual. The omalizumab resin was used to purify human or humanized IgE mAbs in the cultured medium. In brief, 500 ml of the cultured medium was passed through 1 ml of omalizumab resin. The resin was washed with 10 ml of PBS and eluted with 5 ml of elution buffers (0.1 M glycine, pH 3.0) followed by neutralizing with 0.5 ml of Tris buffers (1 M Tris, pH 9.0). Buffers of the purified antibodies were exchanged to PBS with Amicon Ultra-15 devices (Millipore). A human IgE mAb was also purified from the cultured medium of U266 myeloma cells (ATCC). Sizes of the purified U266 IgE and three humanized IgE mAbs were analyzed by SDS-polyacrylamide gel electrophoresis (FIG. 9C).

### 10. Sensitization of RBL-SX38 Cells and β-Hexosaminidase Release Assays with Defined Protein Component-Specific Humanized IgE mAbs

Humanized IgE hybridomas specific for ovalbumin (Sigma-Aldrich) were prepared and purified by following the procedures described in the previous example. Rat basophilic leukemia cells (RBL SX-38, a gift from Dr. Jean P. Kinet) expressing the alpha, beta, and gamma chains of human FcεRI were used to test the IgE sensitization and receptor activation by measuring the β-hexosaminidase activity released after cell degranulation. RBL SX-38 cells were seeded in 200 μl of the culture medium (1×10⁵ cells/well) in a 96-well plate overnight in a 37° C. incubator. On the next day, the medium was removed after centrifugation at 300×g for 5 min and cells were resuspended in 100 μl of pre-warmed culture medium with purified U266 IgE or one of the humanized IgE mAbs at 1 μg/ml. After incubation at 37° C. for 2 hrs, cells were washed twice with 200 μl of Tyrode's buffer (135 mM NaCl, 5 mM KCl, 5.6 mM glucose, 1.8 mM CaCl₂, 1 mM MgCl₂, 20 mM HEPES, and 0.5 mg/ml BSA, pH 7.3) and then 100 pa of pre-warmed Tyrode's buffer containing different concentrations of ovalbumin or papain were added to test the activation of IgE-sensitized FcεRI. Goat total IgG was used as a negative control of non-activation antibody and polyclonal goat anti-human IgE (Bethyl Laboratories) was used to activate the IgE-sensitized FcεRI. After incubation at 37° C. for 1 hour, the plate was centrifuged at 300×g for 10 min and 50 μl of the supernatant in each well was transferred into a 96-well black OptiPlate™ (Perkin-Elmer, Wellesley, Mass.). The assay solution {0.1 M citric acid with 80 μM of 4-MUG (4-methyl-umbelliferyl-N-acetyl-β-d-glucosaminide), pH 4.5} with equal volume (500 was added into each well for enzymatic reaction of β-hexosaminidase. The plate was shaken shortly and incubated at 37° C. with 8% CO₂ for 1 hour. The reaction was terminated by adding 100 μl of glycine buffer (0.2 M glycine, 0.2 M NaCl, pH 10.7) into wells. The fluorescence intensity of each well was measured by using a Victor 3 fluorescence reader (Perkin-Elmer) at the wavelengths of excitation 355 nm and emission 460 nm. The β-hexosaminidase activity of cells lysed with 1% Triton X-100 was served as the maximum release (100%) of RBL SX-38 cells. The spontaneous release was determined by RBL SX-38 cells sensitized with the IgE mAbs only. The percentage of β-hexosaminidase release was calculated by the following equation: [100×(experimental release−spontaneous release)/(maximum release−spontaneous release)].

The results showed that the humanized IgE mAbs bound to human FcεRI on RBL SX-38 cells well and triggered the β-hexosaminidase release with polyclonal anti-human IgE antibodies effectively as the human IgE control (FIG. 9). Papain and ovalbumin can trigger the β-hexosaminidase release of RBL SX-38 cells sensitized with papain- and ovalbumin-specific humanized IgE mAbs, respectively (FIG. 9). The extent of the β-hexosaminidase release of ovalbumin-specific humanized IgE-sensitized RBL SX-38 cells was proportional to the concentration of ovalbumin added (FIG. 9).

### BRIEF DESCRIPTION OF THE FIGURES

FIG. 1A The BAC clones containing gene exons encoding four mouse immunoglobulin Cγ chains (RP24-258E20) and the mouse Cκ chain (RP23-5905), respectively. The F replicon provided a replication origin of BAC DNA and cmr was a chloramphenicol-resistant gene. FIG. 1B Steps to construct the DNA stretch of human Cε gene (˜4,000 bp) with two overhangs of the mouse Cγ1 gene (˜2,000 bp for each overhang).

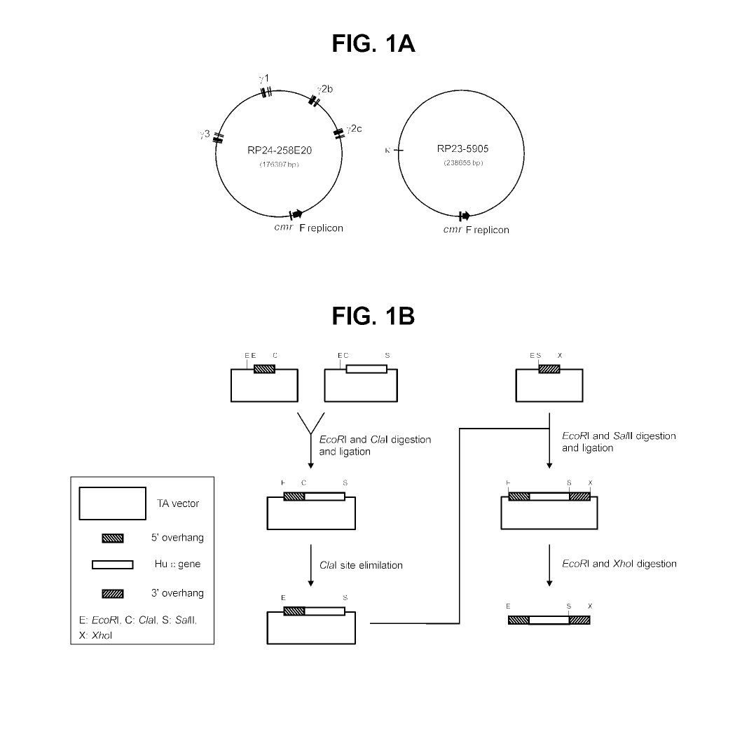

FIG. 2 Replacement of the mouse immunoglobulin Cγ1-encoding gene by the human Cε-encoding gene. A neomycin-resistant gene cassette (neo) was inserted in the 3′ down-stream region of Cγ1 membrane exons.

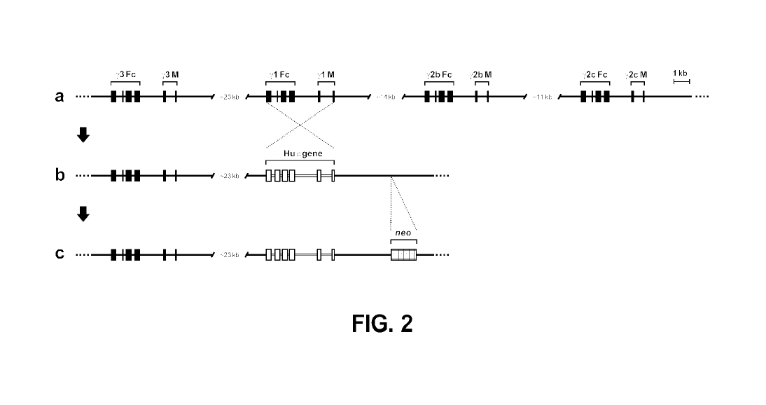

FIG. 3 Replacement of the gene exon encoding the mouse Cκ chain by that encoding the human Cκ chain. A neomycin-resistant gene cassette (neo) was inserted in the 3′ down-stream region of the Cκ exon.

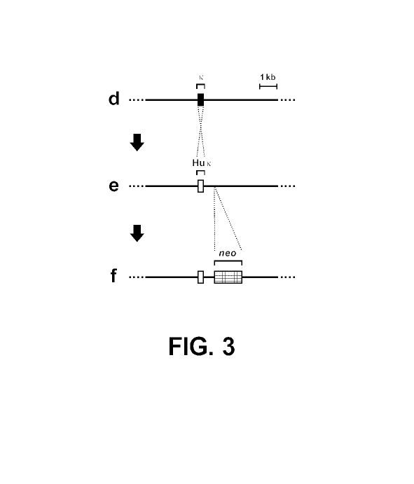

FIG. 4A The primers and the hybridization probe for studying the human Cε chain transgene. B, BamHI; Nt, NotI; S, SacII. FIG. 4B The primers for studying the human Cκ chain transgene. Nr, NruI. FIG. 4C Genotyping of the human Cε and Cκ chain transgenes with PCR. FIG. 4D Southern blotting analyses of the human Cε chain transgene.

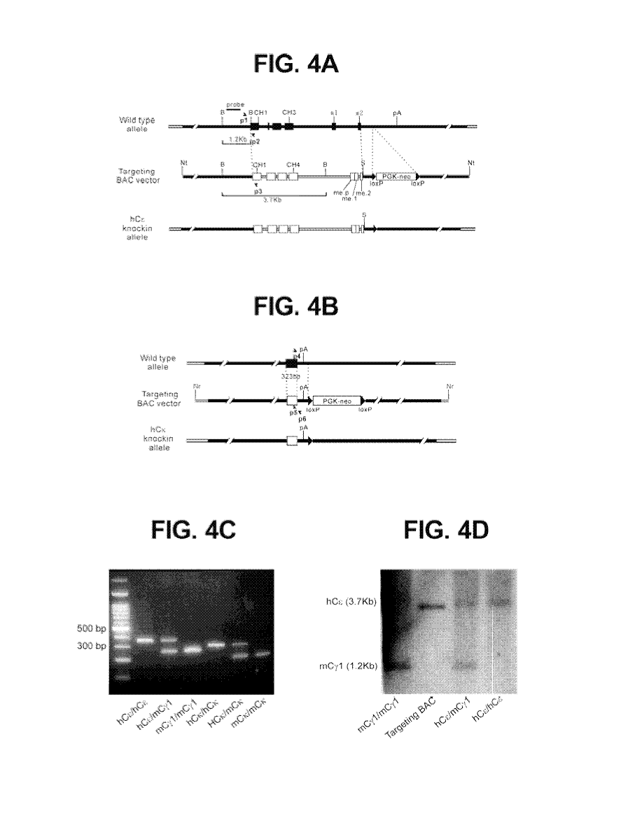

FIG. 5A Measurement of mouse Cγ1 mRNA in mouse spleens of the three genotypes with real-time qPCR. FIG. 5B Measurement of human Cε mRNA in mouse spleens of the three genotypes with real-time qPCR.

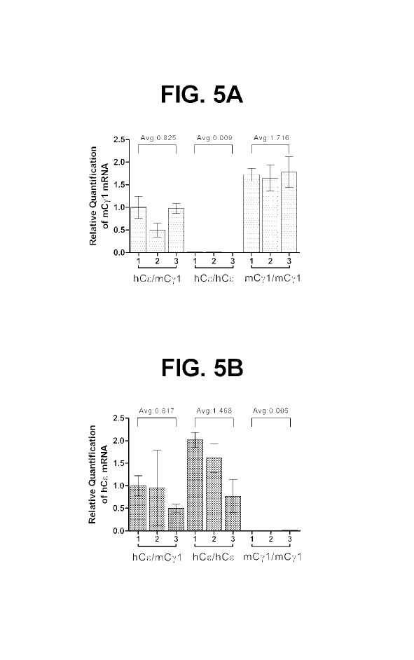

FIG. 6A Measurement of mouse total IgG- and IgG1-secreting B cells in mouse spleens of the three genotypes. MuIgG1, mouse IgG1. FIG. 6B Measurement of the humanized IgE- and mouse IgE-secreting B cells in mouse spleens of the three genotypes. HuIgE, humanized IgE; MuIgE, mouse IgE.

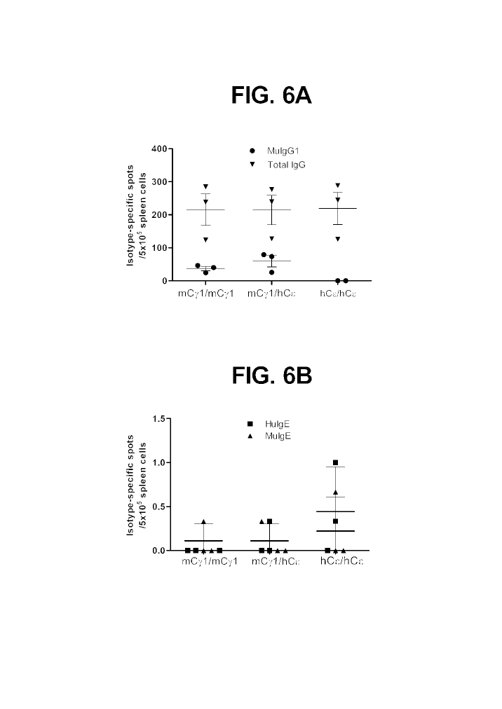

FIG. 7 Measurement of serum levels of different Ig isotypes in papain-immunized mice of the three genotypes with ELISA.

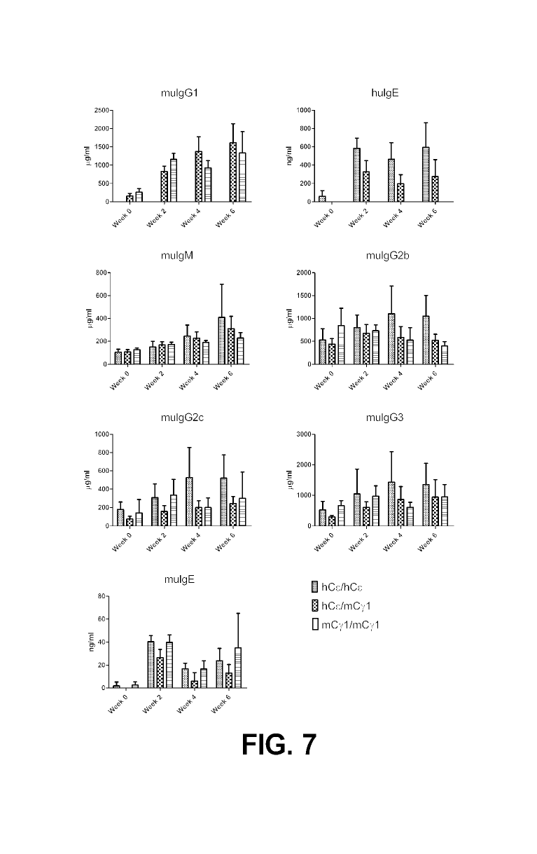

FIG. 8A Binding activity of three identified papain-specific humanized IgE mAbs with ELISA. OVA, ovalbumin; HSA, human serum albumin; BSA, bovine serum albumin. FIG. 8B Isotype determination of light chains of the three humanized IgE mAbs with ELISA. FIG. 8C Analysis of three purified humanized IgE mAbs in a 12% polyacrylamide gel. Lane M, marker; lane 1, the human IgE mAb produced by U266 myeloma cells; lane 2, MAb 106; lane 3, Mab 15G10; lane 4, Mab 34C2; lane 5, polyclonal human IgG.

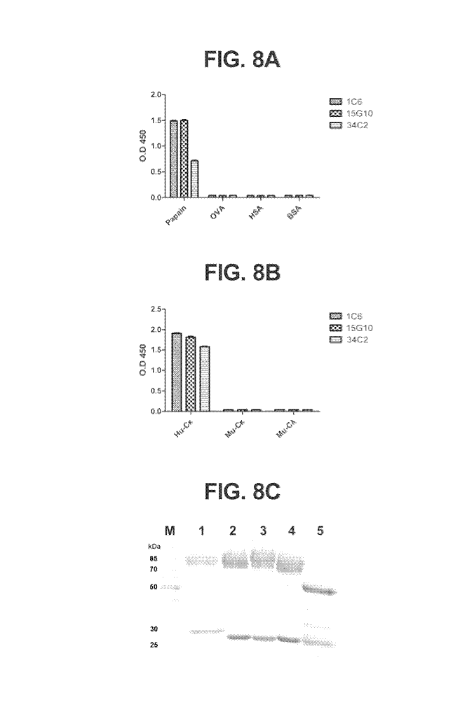

FIG. 9 Determination of β-hexosaminidase release of RBL-SX38 cells sensitized with human IgE and the humanized IgE mAbs. HuIgE, the human IgE mAb produced by U266 myeloma cells; MAb 106, a papain-specific humanized IgE; MAb 8G9, an ovalbumin-specific humanized IgE.

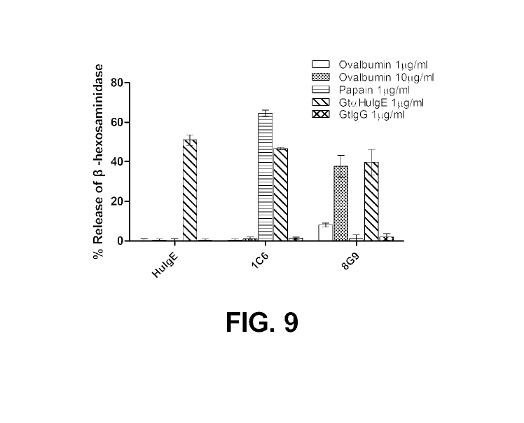

### REFERENCES CITED

Misagh S, Zarrin A, U.S. patent application Ser. No. 13/255,226 (2010) Hyper IgE Animal Model with Enhanced Immunoglobulin Heavy Chain Class Switching to C-epsilon
Böttcher I, Ulrich M, Hirayama N, and Ovary Z (1980) Int. Archs Allergy Immun 61, 248-250
Bohn A, König W (1982) Generation of monoclonal murine anti-DNP-IgE, IgM and IgG1 antibodies: biochemical and biological characterization. Immunology 47, 297-311.
Chaudhuri J, Alt F W (2004) Class switch recombination: Interplay of transcription, DNA deamination and DNA repair. Nat Rev Immunol 4, 541-52
Elliot D, Summers R W, Weinstock J V (2007) Helminths as governors of immune-mediated inflammation. Int'l J Parasit 37, 457-64
Falcone F H, Pritchard, D I (2005) Parasite role reversal: worms on trial. Trends Parasit 21, 157-60
Hanashiro K, Nakamura M, Tamaki N, Kosugi T (1996) Production of a Monoclonal Dinitrophenyl-Specific Rat IgE and Establishment of an IgE Capture ELISA for Estimating the Concentration of Rat IgE Antibodies to Dinitrophenyl-Ascaris suum. Int Arch Allergy Immunol 110, 371-379
Hellman L (2007) Regulation of IgE homeostasis, and the identification of potential targets for therapeutic intervention. Biomed Pharmacotherapy 61, 34-49
Kaul S, Scheurer S, Danz N, Schicktanz S, Vieths S, Hoffmann A (2003) Monoclonal IgE antibodies against birch pollen allergens: Novel tools for biological characterization and standardization of allergens. J Allergy Clin Immunol 111 1262-1268
Kawahara H, Maeda-Yamamoto M, Hakamata K. (2000) Effective induction and acquisition of human monoclonal IgE antibodies reactive with house-dust mite extracts. J Immunol Methods 233, 33-40.
Kellermann S A, Green L L (2002) Antibody discovery: the use of transgenic mice to generate human monoclonal antibodies for therapeutics. Curr Opin Biotechnol 13, 593-7
Lonberg N (2005) Human antibodies from transgenic animals. Nat Biotechnol 23, 1117-25
Lonberg N (2008) Human monoclonal antibodies from transgenic mice. Experim Pharmacol 181, 69-97
Lübben W, Turqueti-Neves A, Okhrimenko A, Stöberl C, Schmidt V, Pfeffer K, Dehnert S, Wünsche S, Storsberg S, Paull S, Bauer S, Riethmüller S, Voehringer S, Yu P (2013) IgE knock-in mice suggest a role for high levels of IgE in basophil-mediated active systemic anaphylaxis. Eur J Immunol 43, 1231-1242
Misaghi S, Senger K, Sai T, Qu Y, Sun Y, Hamidzadeh K, Nguyen A, Jin Z, Zhou M, Yan D, Lin W Y, Lin Z, Lorenzo M N, Sebrell A, Ding J, Xu M, Caplazi P, Austin C D, Balazs M, Roose-Gintia M, DeForge L, Warming S, Lee W P, Dixit V M, Zarrin A A. (2013) Polyclonal hyper-IgE mouse model reveals mechanistic insights into antibody class switch recombination. Proc Natl Acad Sci 110, 15770-15775
Moncayo A L, Cooper P J (2006) Geohelminth infections: Impact on allergic diseases. Int'l J Biochem Cell Biol 38, 1031-5
Oshiba A, Hamelmann E, Takeda K, Bradley K L, Loader J E, Larsen G L, Gelfand ε W (1996) Passive transfer of immediate hypersensitivity and airway hyperresponsiveness by allergen-specific immunoglobulin (Ig) ε and IgG1 in mice. J Clin Invest 97, 1398-1408
Pan-Hammarstroem Q, Zhao Y, Hammarstroem L (2007) Class switch recombination: A comparison between mouse and human. Adv Immunol 93, 1-61
Stavnezer J, Amemiya C T (2004) Evolution of isotype switching. Semin Immunol 16, 257-75
Schuurman J, Perdok G J, Lourens T E, Parren P W, Chapman M D, Aalberse R C (1996) Production of a mouse/human chimeric IgE monoclonal antibody to the house dust mite allergen Der p 2 and its use for the absolute quantification of allergen-specific IgE. J Allergy Clin Immunol 99, 545-50

1. A transgenic animal, in whose genome the gene segment encoding CH1-CH2-CH3-M1-M2 of one of the animal's endogenous immunoglobulins of Cγ is replaced by the gene segment encoding CH1-CH-2-CH3-CH4-M1-M2 of human immunoglobulin Cε.

2. A transgenic animal of claim 1, in which the animal is a mouse, rat, or rabbit.

3. A transgenic animal of claim 1, in which the animal is a mouse and the Cγ is Cγ1.

4. A transgenic mouse of claim 3, in which the mouse is further crossed with a transgenic mouse, in whose genome the mouse's endogenous Cκ constant region coding sequence is replaced by the human immunoglobulin Cκ constant region coding sequences.

5. A method for producing serum or antigen-specific antiserum containing humanized IgE by using a transgenic animal, in whose genome the gene segment encoding CH1-CH2-CH3-M1-M2 of one of the animal's endogenous immunoglobulins of Cγ is replaced by the gene segment encoding CH1-CH2-CH3-CH4-M1-M2 of human immunoglobulin Cε; for the method of producing antigen-specific antiserum, the animal is immunized with the specific antigen.

6. A method for producing serum or antigen-specific antiserum containing humanized IgE of claim 5, wherein the transgenic animal is a mouse, rat, or rabbit.

7. A method for producing serum or antigen-specific antiserum containing humanized IgE of claim 5, wherein the animal is a mouse and the Cγ is Cγ1.

8. A method for producing serum or antigen-specific antiserum containing humanized IgE of claim 7, wherein the mouse strain is further crossed with a transgenic mouse strain, in whose genome the mouse's endogenous Cκ constant region sequence is replaced by the human immunoglobulin Cκ constant region sequence; the homozygous mouse strain with both transgenic human Cε and Cκ is used as the host for the production of serum or antigen-specific antiserum.

9. A method of preparing antigen-specific humanized IgE-secreting hybridomas by using the lymphocytes of a transgenic animal, in whose genome the gene segment encoding CH1-CH2-CH3-M1-M2 of one of the animal's endogenous immunoglobulins of Cγ is replaced by the gene segment encoding CH1-CH2-CH3-CH4-M1-M2 of human immunoglobulin Cε; the animal is immunized with the specific antigen.

10. A method of preparing antigen-specific humanized IgE-secreting hybridomas of claim 9, wherein the transgenic animal is a mouse, rat, or rabbit

11. A method of preparing antigen-specific humanized IgE-secreting hybridomas of claim 9, wherein the animal is a mouse and the Cγ is Cγ1.

12. A method of preparing antigen-specific humanized IgE-secreting hybridomas of claim 11, wherein the mouse strain is further crossed with a transgenic mouse strain, in whose genome the mouse's endogenous Cκ constant region sequence is replaced by the human immunoglobulin Cκ constant region sequence; the homozygous mouse strain with both transgenic human Cε and Cκ is used as the immunization host with the specific antigen for the preparation of hybridomas.
